(19) United States
(12) Patent Application Publication
(10) Pub. No.: US 2023/0149296 A1
(43) Pub. Date: May 18, 2023

(54) DRUG DELIVERY SYSTEM FOR THE DELIVERY OF ANTIVIRAL AGENTS AND CONTRACEPTIVES

(71) Applicants: Nicole M. BEELER, Rahway, NJ (US); Morgan B. GILES, Rahway, NJ (US); Seth P. FORSTER, West Point, PA (US); Stephanie Elizabeth BARRETT, Rahway, NJ (US); Athanas KOYNOV, Rahway, NJ (US); Ryan TELLER, Bend, OR (US); Merck Sharp & Dohme LLC, Rahway, NJ (US)

(72) Inventors: Morgan B. Giles, Weehawken, NJ (US); Seth P. Forster, Fort Washington, PA (US); Stephanie Elizabeth Barrett, Cranford, NJ (US); Athanas Koynov, Metuchen, NJ (US); Ryan S. Teller, Bend, OR (US)

(73) Assignee: Merck Sharp & Dohme LLC, Rahway, NJ (US)

(21) Appl. No.: 17/920,919

(22) PCT Filed: Apr. 30, 2021
(86) PCT No.: PCT/US2021/030032

§ 371 Date: Oct. 24, 2022

Related U.S. Application Data
(60) Provisional application No. 63/020,292, filed on May 5, 2020.

(51) Int. Cl. A61K 9/00; A61K 31/7076; A61K 31/57; A61K 51/12

(52) U.S. Cl. CPC A61K 9/0024; A61K 31/57; A61K 31/7076; A61K 51/1203

(57) ABSTRACT

This invention relates to novel implant drug delivery systems for long-acting delivery of antiviral and contraceptive drugs. These compositions are useful for the treatment or prevention of human immunodeficiency virus (HIV) infection and the prevention of pregnancy.

### BACKGROUND OF THE INVENTION

The development of highly active antiretroviral therapy (HAART) in the mid 1990’s transformed the clinical care of human immunodeficiency virus (HIV) type I infection. HAART regimens have proven to be highly effective treatments, significantly decreasing HIV viral load in HIV-infected patients, thereby slowing the evolution of the illness and reducing HIV-related morbidity and mortality. Yet, the treatment success of HAART is directly related to adherence to the regimen by the patient. Unless appropriate levels of the antiretroviral drug combinations are maintained in the blood, viral mutations will develop, leading to therapy resistance and cross-resistances to molecules of the same therapeutic class, thus placing the long-term efficacy of treatments at risk. Various clinical studies have shown a decline in treatment effectiveness with relatively small lapses in adherence. A study by Musiime found that 81% of patients with more than 95% adherence demonstrated viral suppression, while only 50% of patients who were 80-90% adherent were successful. See, Musiime, S., et al., Adherence to Highly Active Antiretroviral Treatment in HIV-Infected Rwandan Women. PLOS one 2011, 6, (11), 1-6. Remarkably, only 6% of patients that were less than 70% adherent showed improvements in viral markers. Thus, low adherence is a leading cause of therapeutic failure in treatment of HIV-1 infection.

Nonetheless, adherence rates to the HAART regimens continue to be far from optimal. Various characteristics of HAART make adherence particularly difficult. Therapeutic regimens are complex, requiring multiple drugs to be taken daily, often at different times of the day, and many with strict requirements on food intake. Many HAART medications also have unpleasant side effects, including nausea, diarrhea, headache, and peripheral neuropathy. Social and psychological factors can also negatively impact adherence. Patients report that forgetfulness, lifestyle factors, including fear of being identified as HIV-positive, and therapy fatigue over life-long duration of treatment all contribute to adherence lapses.

New HIV treatment interventions aim to improve adherence by reducing the complexity of treatments, the frequency of the dosages, and/or the side effects of the medications. Long-acting injectable (LAI) drug formulations that permit less frequent dosing, on the order of a month or longer, are an increasingly attractive option to address adherence challenges. However, the majority of approved and investigational antiretroviral agents are not well suited for reformulation as long-acting injectable products. In large part, this is due to suboptimal physicochemical properties limiting their formulation as conventional drug suspensions, as well as insufficient antiviral potency resulting in high monthly dosing requirements. Even for cabotegravir or rilpivirine, two drugs being studied as long-acting injectable formulations, large injection volumes and multiple injections are required to achieve pharmacokinetic profiles supportive of monthly dosing. See, e.g., Spreen, W. R., et al., Long-acting injectable antiretrovirals for HIV treatment and prevention. Current Opinion in Hiv and Aids 2013, 8, (6), 565-571; Rajoli, R. K. R., et al., Physiologically Based Pharmacokinetic Modelling to Inform Development of Intramuscular Long-Acting Nanoformulations for HIV. Clinical Pharmacokinetics 2015, 54, (6), 639-650; Baert, L., et al., Development of a long-acting injectable formulation with nanoparticles of rilpivirine (TMC278) for HIV treatment. European Journal of Pharmaceutics and Biopharmaceutics 2009, 72, (3), 502-508; Van ’t Klooster, G., et al., Pharmacokinetics and Disposition of Rilpivirine (TMC278) Nanosuspension as a Long-Acting Injectable Antiretroviral Formulation. Antimicrobial Agents and Chemotherapy 2010, 54, (5), 2042-2050. Thus, novel formulation approaches capable of delivering extended-duration pharmacokinetic characteristics for molecules of diverse physicochemical properties at practical injection volumes and with a limited number of injections are highly desirable.

Contraceptive implants, such as Nexplanon®, are long-term forms of reversible birth control that prevent pregnancy. However, contraceptive implants do not treat or prevent sexually transmitted diseases, such as HIV. An implant that combines reversible birth control with treatment or prevention of HIV infection would be highly desirable.

### SUMMARY OF THE INVENTION

This invention relates to novel implant drug delivery systems for long-acting delivery of antiviral and contraceptive drugs. These compositions are useful for the treatment or prevention of human immunodeficiency virus (HIV) infection and prevention of pregnancy.

### BRIEF DESCRIPTION OF THE DRAWINGS

FIG. 1 is a graph of a Powder X-Ray Diffraction (“PXRD”) pattern of an anhydrate crystalline form of islatravir, generated using the equipment and methods described herein. The graph plots the intensity of the peaks as defined by counts per second versus the diffraction angle 2 theta (2Θ) in degrees.

### DETAILED DESCRIPTION OF THE INVENTION

This invention relates to novel implant drug delivery systems for long-acting delivery of antiviral and contraceptive drugs. The novel implant drug delivery systems comprise a polymer, an antiviral agent and a contraceptive. These implant drug delivery systems are useful for the treatment or prevention of human immunodeficiency virus (HIV) infection and for contraception. The invention further relates to methods of treating and preventing HIV infection and pregnancy with the novel implant drug delivery systems described herein.

The novel implant delivery systems of the invention comprise a biocompatible nonerodible polymer to generate monolithic matrices with dispersed or dissolved drug. The chemical properties of the polymer matrices are tuned to achieve a range of drug release characteristics, offering the opportunity to extend duration of dosing. In an embodiment of the invention, the novel implant delivery systems are compatible with molecules having a broad spectrum of physicochemical properties, including those of high aqueous solubility or amorphous phases which are unsuitable to formulation as solid drug suspensions.

Specifically, this invention relates to novel implant drug delivery systems comprising:
(a) a core comprising a biocompatible nonerodible polymer and (i) islatravir anhydrate, which is present in the core between 10% to 50% by weight, and (ii) etonogestrel, which is present in the core between 25% to 50% by weight, and
(b) a biocompatible nonerodible diffusional barrier comprising a polymer,
   wherein said diffusional barrier has a thickness between 50 µm and 300 µm, wherein said implant drug delivery system is implanted subdermally and (i) islatravir is continually released in vivo at a rate resulting in a plasma concentration of islatravir between 0.02 ng/mL and 300.0 ng/mL, and (ii) etonogestrel is continually released in vivo at a rate resulting in a plasma concentration of etonogestrel between 0.15 ng/mL and 1.2 ng/mL, for a period of six months to thirty-six months. These implant delivery systems are desired and useful for prophylaxis and/or treatment of HIV infection and the prevention of pregnancy, from both compliance and convenience standpoints.

In another aspect, this invention relates to implant drug delivery system comprising an implant, which comprises a core which comprises:
(a) a biocompatible nonerodible polymer,
(b) islatravir anhydrate, which is present in the core between 15% to 35% by weight, and
(c) etonogestrel, which is present in the core between 30% to 40% by weight, wherein said implant drug delivery system is implanted subdermally and islatravir is continually released in vivo at a rate resulting in a plasma concentration of islatravir between 0.02 ng/mL and 300.0 ng/mL, and etonogestrel is continually released in vivo at a rate resulting in a plasma concentration of etonogestrel between 0.15 ng/mL and 1.2 ng/mL, for a period of six months to thirty-six months.

As used herein, the term “biocompatible nonerodible polymer” refers to polymeric materials that are sufficiently resistant to degradation (both chemical and physical) in the presence of biological systems. Biocompatible nonerodible polymers are sufficiently resistant to chemical and/or physical destruction by the environment of use such that the polymer remains essentially intact throughout the release period. The nonerodible polymer is generally hydrophobic so that it retains its integrity for a suitable period of time when placed in an aqueous environment, such as the body of a mammal, and stable enough to be stored for an extended period before use. The nonerodible polymers useful in the invention remain intact in vivo for extended periods of time, typically months or years. Release of dissolved drug from crystalline particles encapsulated in the polymer occurs over time via diffusion through the polymer or through channels and pores formed in the polymer matrix in a sustained manner. The release rate can be altered by modifying the percent drug loading, porosity of the polymer, structure of the implantable device, or hydrophobicity of the polymer, or by adding a diffusional barrier to the exterior of the implantable device.

Accordingly, any polymer that cannot be absorbed by the body can be used to manufacture the implant drug delivery systems of the instant invention. Biocompatible nonerodible polymers of the instant invention include, but are not limited to, ethylene vinyl acetate copolymer (EVA), poly(urethane), silicone, crosslinked poly(vinyl alcohol), poly(hydroxy ethylmethacrylate), acyl substituted cellulose acetates, partially hydrolyzed alkylene-vinyl acetate copolymers, completely hydrolyzed alkylene-vinyl acetate copolymers, unplasticized polyvinyl chloride, crosslinked homopolymers of polyvinyl acetate, crosslinked copolymers of polyvinyl acetate, crosslinked polyesters of acrylic acid, crosslinked polyesters of methacrylic acid, polyvinyl alkyl ethers, polyvinyl fluoride, polycarbonate, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, chlorosulphonated polylefins, and combinations thereof. In a class of the invention, the biocompatible nonerodible polymer is poly(urethane). In a subclass of the invention, the biocompatible nonerodible polymer is hydrophobic poly(urethane).

In another class of the invention, the biocompatible nonerodible polymer is a combination of poly(urethane) and ethylene vinyl acetate copolymer (EVA).

In another class of the invention, the biocompatible nonerodable polymer is ethylene vinyl acetate copolymer (EVA). In a subclass of the invention, the biocompatible nonerodible polymer is selected from the group consisting of ethylene vinyl acetate copolymer (9% vinyl acetate), ethylene vinyl acetate copolymer (15% vinyl acetate), ethylene vinyl acetate copolymer (28% vinyl acetate), and ethylene vinyl acetate copolymer (33% vinyl acetate). In a further subclass of the invention, the biocompatible nonerodible polymer is ethylene vinyl acetate copolymer (9% vinyl acetate). In a further subclass of the invention, the biocompatible nonerodible polymer is ethylene vinyl acetate copolymer (15% vinyl acetate).

In a class of the invention, the biocompatible nonerodible polymer in the core and the polymer of the biocompatible nonerodible diffusional barrier are the same polymer, or the same combination of polymers. In a subclass of the invention, the biocompatible nonerodible polymer in the core and the polymer of the biocompatible nonerodible diffusional barrier are both poly(urethane). In another subclass of the invention, the biocompatible nonerodible polymer in the core and the polymer of the biocompatible nonerodable diffusional barrier are both ethylene vinyl acetate.

As used herein, the term “diffusional barrier” refers to a barrier that is permeable to the drug and is placed over or envelops at least a portion of the core to further regulate the rate of release. For example, a biocompatible nonerodible polymeric material without drug, or a biocompatible nonerodible polymeric material with a lower drug loading than the remainder of the implant delivery system, may be used as the diffusional barrier. The diffusional barrier may be formed, for example, by co-extrusion with the core, by injection molding, or other ways known in the art.

The diffusional barriers of the instant invention comprise hydrophilic polymers or hydrophobic polymers with a soluble filler. In an embodiment of the invention, the diffusional barriers can include additives to increase the hydrophilicity of the diffusional barrier and therefore modulate the release of etonogestrel and islatravir. Suitable additives can include, but are not limited to, polyethylene glycol, citric acid, and poloxamers.

Suitable polymers for use in the diffusional barriers of the instant invention include, but are not limited to, ethylene vinyl acetate copolymer (EVA), silicone, crosslinked poly(vinyl alcohol), unplasticized polyvinyl chloride, crosslinked homopolymers of polyvinyl acetate, crosslinked copolymers of polyvinyl acetate, crosslinked polyesters of acrylic acid, crosslinked polyesters of methacrylic acid, polyvinyl alkyl ethers, polyvinyl fluoride, polycarbonate, polyamide, polysulphones, styrene acrylonitrile copolymers, crosslinked poly(ethylene oxide), poly(alkylenes), poly(vinyl imidazole), poly(ethylene terephthalate), poly(urethane), poly(hydroxy ethylmethacrylate), acyl substituted cellulose acetates, partially hydrolyzed alkylene-vinyl acetate copolymers, completely hydrolyzed alkylene-vinyl acetate copolymers, poly(esters), polyphosphazenes, chlorosulphonated polylefins, and combinations thereof. In a class of the invention, the diffusional barrier is selected from the group consisting of hydrophilic poly(urethane), poly(hydroxy ethylmethacrylate), acyl substituted cellulose acetates, partially hydrolyzed alkylene-vinyl acetate copolymers, completely hydrolyzed alkylene-vinyl acetate copolymers, poly(esters), polyphosphazenes, chlorosulphonated polylefins, and combinations thereof. In a subclass of the invention, the diffusional barrier comprises hydrophilic poly(urethane). In a further subclass of the invention, the hydrophilic poly(urethane) has a water uptake of between 1% and 100% by weight. In a further subclass of the invention, the poly(urethane) has a water uptake of between 1% and 20% by weight.

In an embodiment of the invention, the diffusional barrier has a thickness between 50 µm and 300 µm. In a class of the embodiment, the diffusional barrier has a thickness between 50 µm and 200 µm. In another class of the embodiment, the diffusional barrier has a thickness between 100 µm and 200 µm.

In an embodiment of the invention, the diffusional barrier contains an antiviral drug, etonogestrel or both an antiviral drug and etonogestrel. In a class of the embodiment, the diffusional barrier comprises islatravir anhydrate. In another class of the embodiment, the diffusional barrier comprises etonogestrel. In another class of the embodiment, the diffusional barrier comprises islatravir anhydrate and etonogestrel.

In one embodiment of the inveniton, the term “dispersed or dissolved in the biocompatible nonerodible polymer” refers to the drugs and polymer being mixed and then hotmelt extruded.

As used herein, the term “continually released” refers to the drugs being released from the biocompatible nonerodible polymer at a sufficient rate over extended periods of time to achieve a desired therapeutic or prophylactic concentration. The implant drug delivery systems of the instant invention generally exhibit linear release kinetics for the drugs in vivo, sometimes after an initial burst. The islatravir anhydrate in the core converts to islatravir monohydrate upon exposure to aqueous media, such as blood and plasma, followed by dissolution and release. When measuring the concentration in vivo, it is the concentration of the dissolved molecule, islatravir, that is measured.

The terms “treating” or “treatment” as used herein with respect to an HIV viral infection or AIDS, includes inhibiting the severity of HIV infection or AIDS, i.e., arresting or reducing the development of the HIV infection or AIDS or its clinical symptoms; or relieving the HIV infection or AIDS, i.e., causing regression of the severity of HIV infection or AIDS or its clinical symptoms.

The terms “preventing,” or “prophylaxis,” as used herein with respect to an HIV viral infection or AIDS, refers to reducing the likelihood or severity of HIV infection or AIDS.

The term “contraception” as used herein refers to the prevention of pregnancy.

Optionally, the novel implant delivery systems of the instant invention can further comprise a radiopaque component. The radiopaque component will cause the implant to be X-ray visible. The radiopaque component can be any such element known in the art, such as barium sulphate, titanium dioxide, bismuth oxide, tantalum, tungsten or platinum. In a specific embodiment, the radiopaque component is barium sulphate.

In one embodiment, the radiopaque material is 1% to 30% by weight. In another embodiment, the radiopaque material is 1% to 20% by weight. In another embodiment, the radiopaque material is 4% to 25% by weight. In further embodiment, the radiopaque material is 6% to 20% by weight. In another embodiment, the radiopaque material is about 4% to 15% by weight. In another embodiment, the radiopaque material is about 8% to 15% by weight.

The radiopaque material does not affect the release of islatravir anhydrate or etonogestrel from the implant.

The novel implant delivery systems of the invention comprise antiviral agents and contraceptives. Suitable antiviral agents include anti-HIV agents.

An “anti-HIV agent” is any agent which is directly or indirectly effective in the inhibition of HIV reverse transcriptase or another enzyme required for HIV replication or infection, or the prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. Suitable anti-viral agents for use in implant drug delivery systems described herein include, for example, those listed in Table A as follows:

Antiviral Agents for Preventing HIV infection or AIDS

| | |
|---|---|
| Name | Type |
| abacavir, ABC, Ziagen® | nRTI |
| abacavir +lamivudine, Epzicom® | nRTI |
| abacavir + lamivudine + zidovudine, Trizivir® | nRTI |
| amprenavir, Agenerase® | PI |
| atazanavir, Reyataz® | PI |
| AZT, zidovudine, azidothymidine, Retrovir® | nRTI |
| Capravirine | nnRTI |
| darunavir, Prezista® | PI |
| ddC, zalcitabine, dideoxycytidine, Hivid® | nRTI |
| ddI, didanosine, dideoxyinosine, Videx® | nRTI |
| ddI (enteric coated), Videx EC® | nRTI |
| delavirdine, DLV, Rescriptor® | nnRTI |
| Doravirine | nnRTI |
| doravirine + lamivudine + tenofovir D F | nnRTI + nRTI |
| efavirenz, EFV, Sustiva®, Stocrin® | nnRTI |
| efavirenz + emtricitabine + tenofovir DF, Atripla® | nnRTI + nRTI |
| emtricitabine, FTC, Emtriva® | nRTI |
| emtricitabine + tenofovir DF, Truvada® | nRTI |
| emvirine, Coactinon® | nnRTI |
| enfuvirtide, Fuzeon® | FI |
| enteric coated didanosine, Videx EC®) | nRTI |
| etravirine, TMC-125 | nnRTI |
| fosamprenavir calcium, Lexiva® | PI |
| GS-6207 | CI |
| indinavir, Crixivan® | PI |
| Islatravir | nRTTI |
| lamivudine, 3TC, Epivir® | nRTI |
| lamivudine + zidovudine, Combivir® | nRTI |
| Lopinavir | PI |
| lopinavir + ritonavir, Kaletra® | PI |
| maraviroc, Selzentry® | EI |
| nelfinavir, Viracept® | PI |
| nevirapine, NVP, Viramune® | nnRTI |
| PPL-100 (also known as PL-462) (Ambrilia) | PI |
| raltegravir, Isentress™ | InI |
| (S)-2-(3-chloro-4-fluorobenzyl)-8-ethyl-10-hydroxy-N,6-dimethyl- | InI |
| ritonavir, Norvir® | PI |
| saquinavir, Invirase®, Fortovase® | PI |
| stavudine, d4T,didehydrodeoxythymidine, Zerit® | nRTI |
| tenofovir DF (DF = disoproxil fumarate), TDF, Viread® | nRTI |
| Tenofovir, hexadecyloxypropyl (CMX-157) | nRTI |
| tipranavir, Aptivus® | PI |
| Vicriviroc | EI |

CI = capsid inhibitor; El = entry inhibitor; FI = fusion inhibitor; InI = integrase inhibitor; PI = protease inhibitor; nRTI = nucleoside reverse transcriptase inhibitor; nnRTI = non-nucleoside reverse transcriptase inhibitor; nRTTI = nucleoside reverse transcriptase translocation inhibitor.

Some of the drugs listed in the table can be used in a salt form; e.g., abacavir sulfate, delavirdine mesylate, indinavir sulfate, atazanavir sulfate, nelfinavir mesylate, saquinavir mesylate.

In certain embodiments the antiviral agents in the implant drug delivery systems described herein are employed in their conventional dosage ranges and regimens as reported in the art, including, for example, the dosages described in editions of the Physicians’ Desk Reference, such as the 63rd edition (2009) and earlier editions. In other embodiments, the antiviral agents in the implant drug delivery systems described herein are employed in lower than their conventional dosage ranges. In other embodiments, the antiviral agents in the implant drug delivery systems described herein are employed in higher than their conventional dosage ranges.

In an embodiment of the invention, the antiviral agent can be an entry inhibitor; fusion inhibitor; integrase inhibitor; protease inhibitor; nucleoside reverse transcriptase inhibitor; or non-nucleoside reverse transcriptase inhibitor. In a class of the invention, the antiviral agent is a nucleoside reverse transcriptase inhibitor.

In an embodiment of the invention, the antiviral agent is a nucleoside reverse transcriptase translocation inhibitor (NRTTI). In a class of the invention, the NRTTI is islatravir. In a subclass of the invention, the NRTTI is islatravir anhydrate.

Islatravir (ISL) is also known as 4′-ethynyl-2-fluoro-2′-deoxyadenosine and EFdA, and has the following chemical structure:

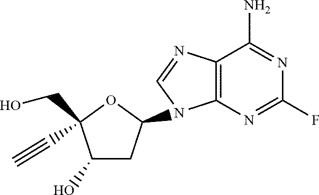 [SMILES: C#C[C@]1(CO)O[C@@H](n2cnc3c(N)nc(F)nc32)C[C@@H]1O]

Production of and the ability of 4′-ethynyl-2-fluoro-2′-deoxyadenosine to inhibit HIV reverse transcriptase are described in PCT International Application WO2005090349, published on Sep. 29, 2005, and U.S. Pat. Application Publication No. 2005/0215512, published on Sep. 29, 2005, both to Yamasa Corporation, both of which are hereby incorporated by reference in their entirety.

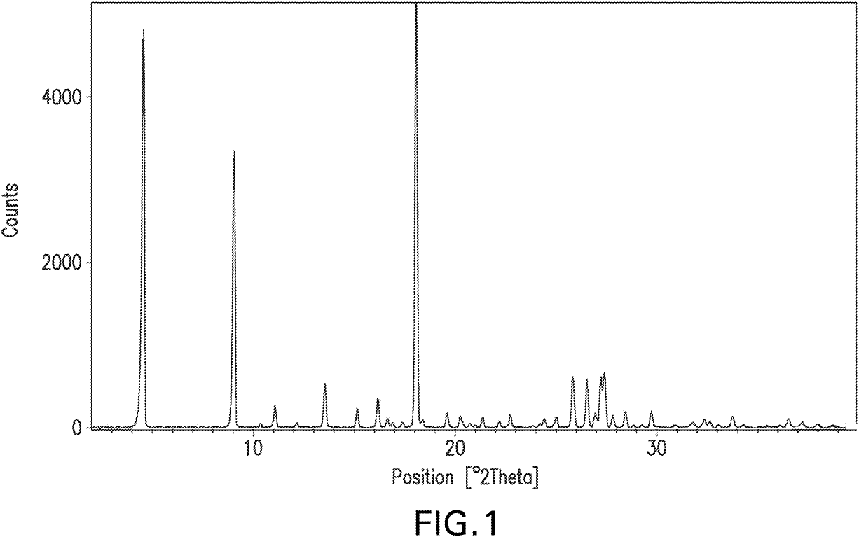

The PXRD pattern for an anhydrate crystalline form of ISL is displayed in FIG. 1 and described in copending application International Application No. PCT/US2019/066436, filed Dec. 16, 2019, which is hereby incorporated by reference in its entirety. Thus, in an aspect of this disclosure, there is provided an anhydrate crystalline form of ISL characterized by a powder x-ray diffraction pattern substantially as shown in FIG. 1. Peak locations (on the 2 theta x-axis) consistent with these profiles are displayed in the table below (+/- 0.2° 2 theta). The locations of these PXRD peaks are characteristic of an anhydrate crystalline form of ISL. Thus, in another aspect, an anhydrate crystalline form of ISL is characterized by a powder x-ray diffraction pattern having each of the peak positions listed in the table below, +/- 0.2° 2-theta.

| Diagnostic Peak Set | Peak Location [°2Th.] (+/- 0.2° 2-theta) | d-spacing [Å] | Relative intensity [%] | Peak No. |
|---|---|---|---|---|
| 2 | 4.48 | 19.73 | 68.9 | 1 |
| 3 | 8.99 | 9.87 | 48.6 | 2 |
| 4 | 10.16 | 8.71 | 15.9 | 3 |
| 3 | 10.39 | 8.51 | 46.0 | 4 |
| 1 | 11.79 | 7.50 | 54.2 | 5 |
| 1 | 12.39 | 7.14 | 36.5 | 6 |
| 1 | 14.70 | 6.03 | 41.2 | 7 |
| 1 | 15.51 | 5.71 | 36.3 | 8 |
| 4 | 15.98 | 5.55 | 17.1 | 9 |
| 4 | 16.64 | 5.33 | 20.1 | 10 |
| 3 | 16.88 | 5.25 | 59.2 | 11 |
| | 17.39 | 5.10 | 13.6 | 12 |
| 2 | 18.09 | 4.91 | 60.3 | 13 |
| | 18.30 | 4.85 | 16.3 | 14 |
| 3 | 20.16 | 4.40 | 67.1 | 15 |
| | 21.69 | 4.10 | 13.4 | 16 |
| 4 | 24.96 | 3.57 | 43.7 | 17 |
| 2 | 25.81 | 3.45 | 100.0 | 18 |
| 2 | 27.42 | 3.25 | 92.7 | 19 |
| | 29.69 | 3.01 | 13.0 | 20 |

Thus, in one aspect, an anhydrate crystalline form of ISL is characterized by a powder x-ray diffraction pattern having each of the peak locations listed in the table above , +/-0.2° 2-theta.

In another aspect, an anhydrate crystalline form of ISL is characterized by a powder x-ray diffraction pattern comprising two or more of the 2-theta values listed in the table above , +/- 0.2° 2-theta.

In another aspect, an anhydrate crystalline form of ISL is characterized by a powder x-ray diffraction pattern comprising three or more of the 2-theta values listed in the table above , +/- 0.2° 2-theta.

In another aspect, an anhydrate crystalline form of ISL is characterized by a powder x-ray diffraction pattern comprising four or more of the 2-theta values listed in the table above , +/- 0.2° 2-theta.

In another aspect, an anhydrate crystalline form of ISL is characterized by a powder x-ray diffraction pattern comprising six or more of the 2-theta values listed in the table above , +/- 0.2° 2-theta.

In another aspect, an anhydrate crystalline form of ISL is characterized by a powder x-ray diffraction pattern comprising nine or more of the 2-theta values listed in the table above , +/- 0.2° 2-theta.

In another aspect, an anhydrate crystalline form of ISL is characterized by a powder x-ray diffraction pattern comprising twelve or more of the 2-theta values listed in the table above , +/- 0.2° 2-theta.

In a further aspect, the PXRD peak locations displayed in the table above and/or FIG. 1 most characteristic of an anhydrate crystalline form of ISL can be selected and grouped as “diagnostic peak sets” to conveniently distinguish this crystalline form from others. Selections of such characteristic peaks are set out in the table above in the column labeled Diagnostic Peak Set.

Thus, in another aspect, there is provided an anhydrate crystalline form of ISL characterized by a powder x-ray diffraction pattern comprising each of the 2-theta values listed in Diagnostic Peak Set 1 in the table above, +/- 0.2° 2-theta.

In another aspect, there is provided an anhydrate crystalline form of ISL characterized by a powder x-ray diffraction pattern comprising each of the 2-theta values listed in Diagnostic Peak Set 2 in the table above, +/- 0.2° 2-theta.

In another aspect, there is provided an anhydrate crystalline form of ISL characterized by a powder x-ray diffraction pattern comprising each of the 2-theta values listed in Diagnostic Peak Set 3 in the table above, +/- 0.2° 2-theta.

In another aspect, there is provided an anhydrate crystalline form of ISL characterized by a powder x-ray diffraction pattern comprising each of the 2-theta values listed in Diagnostic Peak Set 4 in the table above, +/- 0.2° 2-theta.

In another aspect, there is provided an anhydrate crystalline form of ISL characterized by a powder x-ray diffraction pattern comprising each of the 2-theta values listed in Diagnostic Peak Set 1 and any one or more of Diagnostic Peak Set 2, Diagnostic Peak Set 3, and/or Diagnostic Peak Set 4 in the table above, +/- 0.2° 2-theta.

In another aspect, there is provided an anhydrate crystalline Form of ISL characterized by a powder x-ray diffraction pattern comprising each of the 2-theta values listed in Diagnostic Peak Set 2 and any one or more of Diagnostic Peak Set 1, Diagnostic Peak Set 3, and/or Diagnostic Peak Set 4 in the table above, +/- 0.2° 2-theta.

In another aspect, there is provided an anhydrate crystalline form of ISL characterized by a powder x-ray diffraction pattern comprising each of the 2-theta values listed in Diagnostic Peak Set 3 and any one or more of Diagnostic Peak Set 1, Diagnostic Peak Set 2, and/or Diagnostic Peak Set 4 in the table above, +/- 0.2° 2-theta.

In another aspect, there is provided an anhydrate crystalline form of ISL characterized by a powder x-ray diffraction pattern comprising each of the 2-theta values listed in Diagnostic Peak Set 4 and any one or more of Diagnostic Peak Set 1, Diagnostic Peak Set 2, and/or Diagnostic Peak Set 3 in the table above, +/- 0.2° 2-theta.

In another aspect, an anhydrate crystalline form of ISL is characterized by the PXRD spectrum as shown in FIG. 1.

In yet another aspect, anhydrate crystalline form of ISL is characterized by the above described PXRD characteristic peaks and/or the data shown in FIG. 1, alone or in combination with any of the other characterizations of the anhydrate form of ISL described herein.

Etonogestrel (ETO), also known as 3-ketodesogestrel, is a hormone that prevents ovulation. As such it is useful for contraception.

In an embodiment of the implant drug delivery system described herein, etonogestrel is present in the core between 25% to 50% by weight. In a class of the embodiment of the implant drug delivery system described herein, etonogestrel is present in the core between 30% to 45% by weight. In another embodiment of the implant drug delivery system described herein, etonogestrel is present in the core at 30% by weight. In other embodiments, etonogestrel is present in the core at about 35% by weight. In other embodiments, etonogestrel is present in the core at 40% by weight. In an embodiment of the implant drug delivery system descrived herein, islatravir anhydrate is present in the core between 10% to 50% by weight. In a class of the embodiment of the implant drug delivery system described herein, islatravir anhydrate is present in the core between 10% to 35% by weight. In a subclass of the embodiment of the implant drug delivery system described herein, islatravir anhydrate is present in the core at 15% by weight. In another subclass of the embodiment of the implant drug delivery system described herein, islatravir anhydrate is present in the core at about 20% by weight. In another subclass of the embodiment of the implant drug delivery system described herein, islatravir anhydrate is present in the core at about 30% by weight. In another subclass of the embodiment of the implant drug delivery system described herein, islatravir anhydrate is present in the core at 35% by weight.

As an example of the implant drug delivery system described herein, islatravir anhydrate is present in the core at 15% by weight and etonogestrel is present in the core at 35% by weight. As another example of the implant drug delivery system described herein, islatravir anhydrate is present in the core at 15% by weight and etonogestrel is present in the core at 30% by weight. As another example of the implant drug delivery system described herein, islatravir anhydrate is present in the core at 20% by weight and etonogestrel is present in the core at 40% by weight. As an example of the implant drug delivery system described herein, islatravir anhydrate is present in the core at 30% by weight and etonogestrel is present in the core at 30% by weight. As an example of the implant drug delivery system described herein, islatravir anhydrate is present in the core at 35% by weight and etonogestrel is present in the core at 35% by weight.

The implant drug delivery systems of the instant invention may be produced using an extrusion process, wherein ground biocompatible, nonerodible polymer is blended with the antiviral agent, melted and extruded into rod-shaped structures. Rods are cut into individual implantable devices of the desired length, packaged and sterilized prior to use. Other methods for encapsulating therapeutic compounds in implantable polymeric, nonerodible matrices are known to those of skill in the art. Such methods include solvent casting (see U.S. Pat. Nos. 4,883,666, 5,114,719 and 5,601835). One of skill in the art would be able to readily determine an appropriate method of preparing such an implant drug delivery system, depending on the shape, size, drug loading, and release kinetics desired for a particular type of patient or clinical application.

The implant drug delivery systems of the instant invention may be produced using a co-extrusion process of the core and the biocompatible nonerodible diffusional barrier. In an embodiment of the invention, the core and the biocompatible nonerodible diffusional barrier are prepared by co-extrusion, and the co-extrusion is carried out at a temperature between 130° C. and 190° C. In a class of the invention, the biocompatible nonerodible polymer core and the biocompatible nonerodible diffusional barrier are prepared by co-extrusion, and the co-extrusion is carried out at a temperature between 130° C. and 160° C. In another embodiment of the invention, the core is prepared by an injection molding process at temperature between 130 and 190° C. and the biocompatible nonerodable diffusional barrier is carried out by an over-molding process at temperature between 130 and 190° C. and force between 5 and 30 tons. In a subclass of the invention the polymer core is prepared by injection molding, and the injection molding is carried out at temperature between 150 and 160° C. and the biocompatible nonerodable diffusional barrier is carried out by an over-molding process at temperature between 150 and 160° C. and force between 10 and 20 tons.

The size and shape of the implant drug delivery systems may be modified to achieve a desired overall dosage. The implant drug delivery systems of the instant invention are often between 0.5 cm to 10 cm in length. In an embodiment of the invention, the implant drug delivery systems are between 1.5 cm to 5 cm in length. In a class of the embodiment, the implant drug delivery systems are between 2 cm to 5 cm in length. In a subclass of the embodiment, the implant drug delivery systems are between about 2 cm to 4 cm in length. The implant drug delivery systems of the instant invention are often between 0.5 mm to 7 mm in diameter. In an embodiment of the invention, the implant drug delivery systems are between 1.5 mm to 5 mm in diameter. In a class of the embodiment, the implant drug delivery systems are between 2 mm to 5 mm in diameter. In a subclass of the embodiment, the implant drug delivery systems are between about 2 mm to 4 mm in diameter.

The implant drug delivery systems described herein are capable of releasing islatravir at therapeutic concentrations over a period of 21 days, 28 days, 31 days, 4 weeks, 6 weeks, 8 weeks, 12 weeks, one month, two months, three months, four months, five months, six months, seven months, eight months, nine months, ten months, eleven months, twelve months, eighteen months, twenty-four months or thirty-six months. In an embodiment of the invention, islatravir is released at therapeutic concentrations at an average rate of between 0.02-8.0 mg per day. In an embodiment of the invention, islatravir is released at therapeutic concentrations for a duration from between six months and thirty-six months. In a class of the embodiment, islatravir is released at therapeutic concentrations for a duration from between six months and twelve months. In another class of the embodiment, islatravir is released at therapeutic concentrations for a duration from between twenty-four months and thirty-six months. In an embodiment of the invention, islatravir is released at prophylactic concentrations for a duration from between six months and thirty-six months. In a class of the embodiment, islatravir is released at prophylactic concentrations for a duration from between six months and twelve months. In another class of the embodiment, islatravir is released at prophylactic concentrations for a duration from between twenty-four months and thirty-six months.

The implant drug delivery systems described herein are capable of releasing etonogestrel at contraceptive concentrations over a period of 21 days, 28 days, 31 days, 4 weeks, 6 weeks, 8 weeks, 12 weeks, one month, two months, three months, four months, five months, six months, seven months, eight months, nine months, ten months, eleven months, twelve months, eighteen months, twenty-four months or thirty-six months. In an embodiment of the invention, etonogestrel is released at contraceptive concentrations at an average rate of between 25-70 µg per day. In an embodiment of the invention, etonogestrel is released at contraceptive concentrations for a duration from between six months and thirty-six months. In a class of the embodiment, etonogestrel is released at contraceptive concentrations for a duration from between six months and twelve months. In another class of the embodiment, etonogestrel is released at contraceptive concentrations for a duration from between twenty-four months and thirty-six months.

One or more implants can be used to achieve the desired therapeutic, prophylactic or contraceptive dose. In an embodiment of the invention, one or more implants can be used to achieve the therapeutic, prophylactic or contraceptive dose for durations of up to 1 year. In another embodiment of the invention, one or more implants can be used to achieve the therapeutic, prophylactic or contraceptive dose for durations of up to 2 years.

The implant drug delivery systems described herein are capable of releasing islatravir resulting in a plasma concentration of islatravir between 0.02 ng/mL and 300 ng/mL. In an embodiment of the invention, the implant drug delivery systems described herein are capable of releasing islatravir resulting in a plasma concentration of islatravir between 0.02 ng/mL and 300 ng/mL. In a class of the embodiment, the implant drug delivery systems described herein are capable of releasing islatravir resulting in a plasma concentration of islatravir between 0.02 ng/mL and 15.0 ng/mL. In a further class of the embodiment, the implant drug delivery systems described herein are capable of releasing islatravir anhydrate resulting in a plasma concentration of islatravir between 0.02 ng/mL and 8.0 ng/mL. In a subclass of the embodiment, the implant drug delivery systems described herein are capable of releasing islatravir anhydrate resulting in a plasma concentration of islatravir between 0.1 ng/mL and 1.0 ng/mL.

The implant drug delivery systems of the instant invention are capable of releasing etonogestrel with a release rate of 60-70 µg/day in week 5-6, which decreases to approximately 35-45 mcg/day at the end of the first year, to approximately 30-40 µg/day at the end of the second year, and then to approximately 25-30 µg/day at the end of the third year.

In one embodiment, the mean (±SD) maximum serum etonogestrel concentrations are 1200 (± 604) pg/mL which are reached within the first two weeks after insertion. The mean (± SD) serum etonogestrel concentration decreases gradually over time, declining to 202 (± 55) pg/mL at 12 months, 164 (± 58) pg/mL at 24 months, and 138 (± 43) pg/mL at 36 months. In another embodiment, the mean (± SD) maximum serum etonogestrel concentrations are 1145 (± 577) pg/mL and are reached within the first two weeks after insertion. The mean (± SD) serum etonogestrel concentration decrease gradually over time, declining to 223 (±73) pg/mL at 12 months, 172 (± 77) pg/mL at 24 months, and 153 (± 52) pg/mL at 36 months.

The instant invention also comprises implant drug delivery system comprising:
(a) a core comprising a biocompatible nonerodible polymer and (i) islatravir, which is present in the core between 15% to 35% by weight, and (ii) etonogestrel, which is present in the core between 30% to 40% by weight, and optionally comprises
(b) a biocompatible nonerodible diffusional barrier comprising a polymer,
   wherein said diffusional barrier has a thickness between 50 µm and 300 µm, and wherein at day 30, (i) islatravir is released in vitro at a rate resulting in a concentration at of islatravir between 0.020 mg and 0.050 mg, and (ii) etonogestrel is released in vitro at a rate resulting in a concentration of etonogestrel between 0.025 mg and 0.050 mg. In a class of the invention, at day 30, the islatravir is released in vitro at a rate resulting in a concentration at of islatravir between 0.027 mg and 0.042 mg. In another class of the invention, at day 30, the etonogestrel is released in vitro at a rate resulting in a concentration of etonogestrel between 0.031 mg and 0.046 mg.

The following examples are given for the purpose of illustrating the present invention and shall not be construed as being limitations on the scope of the invention.

### Example 1

### Preparation of Implant Drug Delivery Systems Containing 60wt% Islatravir Anhydrate and a Radiopaque Agent

Matrix (core only) implants were prepared using an extrusion injection molded process. Hydrophobic, aliphatic thermoplastic polyurethane and islatravir, anhydrate form, were blended with 60 wt% ISL and 10 wt% Barium Sulfate as a radiopaque agent. The preblend was compounded with a twin screw extruder at temperatures ranging from 150-160° C., screw speed at 50-60 rpm, torque at 40-350 Ncm, and then re-granulated and mixed. The mix was melt extruded with a twin screw extruder at temperatures ranging from 150-160° C., screw speed at 50-60 rpm and torque at 100-350 Ncm to form a 1.8±0.1 mm diameter filament. Extruded filaments were cut to length 50±2 mm and compression molded at 150-160° C. and 10-20 tons. Preformed filaments were then trimmed of flash and cut to final length 40±2 mm to form final matrix implants of islatravir in thermoplastic polyurethane.

Reservoir (matrix + diffusional barrier) implants were prepared using already formed matrix filaments as described above and pre-extruded hydrophilic, swelling thermoplastic polyurethane of 5% or 10% nominal water uptake tubing. Extruded tubing was placed over pre-formed filaments of core material then samples with diffusional barriers were placed in an over-molding machine and compression molded at 180-190° C. and 8-12 tons to bond the core and diffusional barrier, and cut to final length 40±2mm.

### Example 2

### Preparation of Implant Drug Delivery Systems Containing 60wt% Etonogestrel and a Radiopaque Agent

Matrix implants were prepared using an extrusion injection molded process. Hydrophobic, aliphatic thermoplastic polyurethane and etonogestrel, were blended with 60 wt% ETO and 10 wt% Barium Sulfate as a radiopaque agent. The preblend was compounded with a twin screw extruder at temperatures ranging from 150-160° C., screw speed at 50-60 rpm, torque at 30-90 Ncm, and then re-granulated and mixed. The mix was melt extruded with a twin screw extruder at temperatures ranging from 150-160° C., screw speed at 50-60 rpm and torque at 30-60 Ncm to form a 1.8±0.1 mm diameter filament. Extruded filaments were cut to length 50±2 mm and compression molded at 150-160° C. and 10-20 tons. Preformed filaments were then trimmed of flash and cut to final length 40±2 mm to form final matrix implants of etonogestrel in thermoplastic polyurethane.

Reservoir implants were prepared using already formed matrix filaments as described above and pre-extruded hydrophilic, swelling thermoplastic polyurethane of 5% or 10% nominal water uptake tubing. Extruded tubing was placed over pre-formed filaments of core material then samples with diffusional barriers were placed in an over-molding machine and compression molded at 180-190° C. and 8-12 tons to bond the core and diffusional barrier, and cut to final length 40±2 mm.

### Example 3

### Preparation of Implant Drug Delivery Systems Containing 15wt% Islatravir Anhydrate, 35% Etonogestrel, And A Radiopaque Agent

Matrix implants were prepared using an extrusion injection molded process. Hydrophobic, aliphatic thermoplastic polyurethane (TPU), were blended with 15 wt% islatravir anhydrate, 35 wt% etonogestrel and 10 wt% Barium Sulfate as a radiopaque agent. The preblend was compounded with a twin screw extruder at temperatures ranging from 150-160° C., screw speed at 50-60 rpm, torque at 30-60 Ncm, and then re-granulated and mixed. The mix was melt extruded with a twin screw extruder at temperatures ranging from 150-160° C., screw speed at 50-60 rpm and torque at 20-40 Ncm to form a 1.8±0.1 mm diameter filament. Extruded filaments were cut to length 50±2 mm and compression molded at 150-160° C. and 10-20 tons. Preformed filaments were then trimmed of flash and cut to final length 40±2 mm to generate combination implants of islatravir+ etonogestrel in thermoplastic polyurethane.

Reservoir implants were prepared using already formed matrix filaments as described above and pre-extruded hydrophilic, swelling thermoplastic polyurethane of 5% or 10% nominal water uptake tubing as diffusional barrier material. Extruded tubing was placed over pre-formed filaments of core material then samples with diffusional barriers were placed in an over-molding machine and compression molded at 180-190° C. and 8-12 tons to bond the core and diffusional barrier, and cut to final length 40±2 mm.

The in vitro release rate of islatravir and etonogestrel were determined using a Agilent 400-DS USP Apparatus 7 (App7). The full implant was put into a PEEK basket with mesh ends (Agilent part number 33-9046) and was submerged in 10 ml of 1x phosphate buffered saline (PBS) in the incubation vessel of the App7 device. A constant temperature of 37° C. was maintained and monitored by the system. Samples were incubated within the PBS medium at a rate of 20 dips per minute (dpm) with a dipping height of 2 cm. The App7 removed a 1.2 mL sample once each 24 hours and filled it into an HPLC vial. A full media (10 mL) replacement was performed every day (24 h) by the system after the sampling step. Samples were assayed by (U)HPLC (Waters H-Class Acquity with PDA detector). Analysis of a 1 µL volume was performed at 244 nm (ETO) and 261 nm (ISL) with an HSS C18 Gravity column (100 x 2.1 mm, 1.8 µm) maintained at 60° C. The mobile phase A was Water:ACN (95:5 v/v) and mobile phase B with ACN at a flow rate 0.750 mL/min following the gradient listed below.

TABLE 1

Islatravir and etonogestrel chemical stability HPLC gradient method details

| Time (min) | Flow Rate (mL/min) | Mobile phase A (%) | Mobile phase B (%) |
|---|---|---|---|
| 0.00 | 0.750 | 85 | 15 |
| 0.50 | 0.750 | 85 | 15 |
| 1.25 | 0.750 | 25 | 75 |
| 2.50 | 0.750 | 25 | 75 |
| 3.00 | 0.750 | 85 | 15 |
| 5.00 | 0.750 | 85 | 15 |

TABLE 2

Islatravir in vitro release from 15 wt% islatravir:35 wt% etonogestrel in TPU implants as core, with a 5% water uptake or 10% water uptake diffusional barrier at sink conditions; reported as a % release from total [avg = average and std dev = standard deviation]

| Time (days) | 15 wt% ISL:35 wt% ETO in TPU core | | 15 wt% ISL:35 wt% ETO TPU + 5% WU diffusional barrier | | 15 wt% ISL:35% ETO in TPU with 10%WU diffusional barrier | |
|---|---|---|---|---|---|---|
| | avg(%) | std.dev. | avg(%) | std.dev. | avg(%) | std.dev. |
| 1 | 0.576 | 0.065 | 0.078 | 0.014 | 0.105 | 0.006 |
| 2 | 0.229 | 0.031 | 0.059 | 0.005 | 0.082 | 0.007 |
| 3 | 0.164 | 0.021 | 0.056 | 0.001 | 0.073 | 0.006 |
| 4 | 0.137 | 0.016 | 0.055 | 0.001 | 0.066 | 0.006 |
| 5 | 0.119 | 0.015 | 0.052 | 0.004 | 0.061 | 0.007 |
| 6 | 0.106 | 0.015 | 0.049 | 0.002 | 0.056 | 0.004 |
| 7 | 0.096 | 0.012 | 0.048 | 0.003 | 0.053 | 0.004 |
| 8 | 0.089 | 0.012 | 0.046 | 0.005 | 0.050 | 0.004 |
| 9 | 0.083 | 0.011 | 0.045 | 0.005 | 0.048 | 0.003 |
| 10 | 0.079 | 0.010 | 0.042 | 0.004 | 0.046 | 0.003 |
| 11 | 0.074 | 0.009 | 0.042 | 0.005 | 0.044 | 0.002 |
| 13 | 0.069 | 0.009 | 0.038 | 0.004 | 0.040 | 0.002 |
| 14 | 0.065 | 0.007 | 0.039 | 0.006 | 0.040 | 0.002 |
| 15 | 0.063 | 0.007 | 0.037 | 0.004 | 0.038 | 0.002 |
| 16 | 0.061 | 0.007 | 0.037 | 0.005 | 0.037 | 0.001 |
| 17 | 0.058 | 0.006 | 0.037 | 0.006 | 0.036 | 0.001 |
| 18 | 0.057 | 0.006 | 0.035 | 0.004 | 0.035 | 0.001 |
| 19 | 0.055 | 0.006 | 0.035 | 0.005 | 0.034 | 0.001 |
| 20 | 0.053 | 0.005 | 0.035 | 0.006 | 0.033 | 0.001 |
| 21 | 0.052 | 0.006 | 0.035 | 0.007 | 0.033 | 0.001 |
| 22 | 0.050 | 0.005 | 0.032 | 0.006 | 0.031 | 0.001 |
| 23 | 0.049 | 0.005 | 0.031 | 0.005 | 0.031 | 0.000 |
| 24 | 0.047 | 0.005 | 0.031 | 0.005 | 0.030 | 0.001 |
| 25 | 0.046 | 0.005 | 0.030 | 0.004 | 0.029 | 0.000 |
| 26 | 0.047 | 0.005 | 0.031 | 0.006 | 0.029 | 0.000 |
| 27 | 0.045 | 0.004 | 0.030 | 0.005 | 0.029 | 0.000 |
| 28 | 0.045 | 0.004 | 0.030 | 0.006 | 0.028 | 0.000 |
| 29 | 0.044 | 0.004 | 0.031 | 0.008 | 0.028 | 0.000 |
| 30 | 0.042 | 0.003 | 0.030 | 0.006 | 0.027 | 0.000 |
| 31 | 0.042 | 0.003 | 0.028 | 0.004 | 0.027 | 0.000 |
| 32 | 0.041 | 0.004 | 0.026 | 0.003 | 0.026 | 0.000 |

ISL - Islatravir anhydrate
ETO - Etonogestrel
TPU - Thermoplastic PolyUrethane
WU - Water Uptake

TABLE 3

Etonogestrel in vitro release from 15 wt% islatravir:35 wt% etonogestrel in TPU implants as core, with a 5% water uptake or 10% water uptake diffusional barrier at sink conditions; reported as a % release from total [avg = average and std dev = standard deviation]

| Time (days) | 15 wt% ISL:35 wt% ETO in TPU core | | 15 wt% ISL:35 wt% ETO TPU + 5% WU diffusional barrier | | 15 wt% ISL:35% ETO in TPU with 10%WU diffusional barrier | |
|---|---|---|---|---|---|---|
| | avg (%) | std.dev. | avg (%) | std.dev. | avg (%) | std.dev. |
| 1 | 0.054 | 0.005 | 0.033 | 0.007 | 0.035 | 0.0036 |
| 2 | 0.053 | 0.006 | 0.036 | 0.005 | 0.036 | 0.0022 |
| 3 | 0.052 | 0.005 | 0.037 | 0.002 | 0.036 | 0.0014 |
| 4 | 0.051 | 0.004 | 0.036 | 0.002 | 0.035 | 0.0009 |
| 5 | 0.050 | 0.005 | 0.036 | 0.002 | 0.034 | 0.0008 |
| 6 | 0.049 | 0.004 | 0.036 | 0.001 | 0.034 | 0.0007 |
| 7 | 0.049 | 0.005 | 0.035 | 0.001 | 0.034 | 0.0005 |
| 8 | 0.049 | 0.005 | 0.035 | 0.001 | 0.033 | 0.0005 |
| 9 | 0.049 | 0.005 | 0.035 | 0.001 | 0.033 | 0.0002 |
| 10 | 0.049 | 0.005 | 0.035 | 0.001 | 0.033 | 0.0002 |
| 11 | 0.048 | 0.005 | 0.034 | 0.001 | 0.033 | 0.0000 |
| 13 | 0.049 | 0.004 | 0.034 | 0.001 | 0.032 | 0.0003 |
| 14 | 0.048 | 0.004 | 0.035 | 0.001 | 0.033 | 0.0006 |
| 15 | 0.049 | 0.006 | 0.035 | 0.001 | 0.034 | 0.0006 |
| 16 | 0.048 | 0.005 | 0.034 | 0.001 | 0.032 | 0.0002 |
| 17 | 0.048 | 0.005 | 0.034 | 0.001 | 0.032 | 0.0000 |
| 18 | 0.048 | 0.004 | 0.034 | 0.001 | 0.032 | 0.0007 |
| 19 | 0.049 | 0.004 | 0.034 | 0.001 | 0.032 | 0.0004 |
| 20 | 0.048 | 0.004 | 0.034 | 0.001 | 0.032 | 0.0002 |
| 21 | 0.048 | 0.005 | 0.035 | 0.001 | 0.032 | 0.0005 |
| 22 | 0.047 | 0.005 | 0.033 | 0.001 | 0.031 | 0.0000 |
| 23 | 0.046 | 0.004 | 0.033 | 0.001 | 0.031 | 0.0002 |
| 24 | 0.047 | 0.005 | 0.033 | 0.001 | 0.031 | 0.0003 |
| 25 | 0.046 | 0.005 | 0.033 | 0.001 | 0.031 | 0.0003 |
| 26 | 0.046 | 0.005 | 0.033 | 0.001 | 0.031 | 0.0002 |
| 27 | 0.046 | 0.004 | 0.033 | 0.001 | 0.031 | 0.0001 |
| 28 | 0.046 | 0.004 | 0.033 | 0.001 | 0.031 | 0.0002 |
| 29 | 0.046 | 0.005 | 0.033 | 0.001 | 0.031 | 0.0003 |
| 30 | 0.046 | 0.004 | 0.032 | 0.001 | 0.031 | 0.0001 |
| 31 | 0.046 | 0.004 | 0.032 | 0.001 | 0.031 | 0.0001 |
| 32 | 0.046 | 0.005 | 0.033 | 0.001 | 0.031 | 0.0004 |

TABLE 4

Islatravir and etonogestrel in vitro release rates from 15 wt% islatravir:35 wt% etonogestrel in TPU core implants with a 250 µm or 200 µm TPU diffusional barrier

| Sample | ISL Release Rate at day 30 (mg/day) | ETO Release rate at day 30 (mg/day) |
|---|---|---|
| 15 wt% ISL:35% ETO in TPU core | 0.042 | 0.046 |
| 15 wt% ISL:35 wt% ETO in TPU with 5%WU diffusional barrier | 0.030 | 0.032 |
| 15 wt% ISL:35 wt% ETO in TPU with 10%WU diffusional barrier | 0.027 | 0.031 |

### Example 4

### Pharmacokinetic Studies of Islatravir And Etonogestrel

The in-vivo release profile of islatravir and etonogestrel was assessed in an exploratory pharmacokinetic study in rats following a single subcutaneous placement of a solid Long Acting Parenteral (LAP) formulation of islatravir and etonogestrel. The duration of the study was 60 days. Both matrix implants and reservoir implants with a 10% WU sheath (as described above) were implanted. Each dose group consisted of four male animals. Four different drug load combinations were studied in each formulation case.

Projected islatavir-etonogestrel in-vivo release rates in humans were estimated based on the rat pharmacokinetic data. The daily release rate (in ug/day) up to day 60 was estimated via deconvolution of the observed plasma concentration profiles against observed intravenous data for each compound, correcting for the respective implant and intravenous doses. The projected in-vivo release rates in humans are shown in the tables below.

TABLE 5

In-vivo release of islatravir from implants in humans (model) - deconvoluted from rat pharmacokinetic studies

| | 15%DL Islatravir 35%DL Etonogestrel | 20%DL Islatravir 40%DL Etonogestrel | 30%DL Islatravir 30%DL Etonogestrel | 35%DL Islatravir 35%DL Etonogestrel | 15%DL Islatravir 35%DL Etonogestrel | 20%DL Islatravir 40%DL Etonogestrel | 30%DL Islatravir 30%DL Etonogestrel | 35%DL Islatravir 35%DL Etonogestrel |
|---|---|---|---|---|---|---|---|---|
| Days | Core-Diffusional Barrier | | | | Matrix | | | |
| | Daily Release, mg/day | Daily Release, mg/day | Daily Release, mg/day | Daily Release, mg/day | Daily Release, mg/day | Daily Release, mg/day | Daily Release, mg/day | Daily Release, mg/day |
| 0.04 | 0.52 | 0.60 | 0.82 | 2.96 | 2.60 | 3.41 | 9.00 | 18.12 |
| 0.08 | 0.37 | 0.47 | 0.69 | 2.07 | 1.85 | 2.89 | 6.82 | 15.25 |
| 0.17 | 0.22 | 0.35 | 0.44 | 1.27 | 1.12 | 1.54 | 3.83 | 10.89 |
| 1.00 | 0.20 | 0.28 | 1.03 | 0.64 | 0.69 | 1.28 | 2.47 | 6.81 |
| 2.00 | 0.15 | 0.22 | 0.22 | 0.44 | 0.56 | 1.06 | 2.23 | 5.35 |
| 3.00 | 0.15 | 0.19 | 0.24 | 0.38 | 0.51 | 0.93 | 1.84 | 4.12 |
| 4.00 | 0.13 | 0.16 | 0.24 | 0.33 | | 0.81 | 1.55 | 3.66 |
| 7.00 | 0.11 | 0.14 | 0.19 | 0.26 | 0.37 | 0.57 | 1.01 | 2.48 |
| 10.00 | 0.10 | 0.12 | 0.17 | 0.22 | 0.27 | 0.38 | 0.71 | 1.39 |
| 15.00 | 0.08 | 0.10 | 0.16 | 0.21 | 0.21 | 0.30 | 0.57 | 1.16 |
| 17.00 | 0.07 | 0.10 | 0.18 | 0.23 | 0.20 | 0.28 | 0.48 | 0.91 |
| 22.00 | 0.08 | 0.11 | 0.15 | 0.18 | 0.19 | 0.27 | 0.45 | 0.83 |
| 24.00 | 0.07 | 0.09 | 0.15 | 0.18 | 0.15 | 0.24 | 0.35 | 0.54 |
| 29.00 | 0.09 | 0.11 | 0.15 | 0.17 | 0.14 | 0.20 | 0.31 | 0.46 |
| 31.00 | 0.07 | 0.08 | 0.12 | 0.13 | 0.11 | 0.17 | 0.27 | 0.36 |
| 36.00 | 0.08 | 0.09 | 0.15 | 0.15 | 0.08 | 0.14 | 0.23 | 0.28 |
| 38.00 | 0.06 | 0.08 | 0.14 | 0.15 | 0.11 | 0.14 | 0.22 | 0.21 |
| 43.00 | 0.06 | 0.08 | 0.15 | 0.16 | 0.10 | 0.13 | 0.19 | 0.20 |
| 45.00 | 0.05 | 0.07 | 0.11 | 0.12 | 0.09 | 0.13 | 0.18 | 0.13 |
| 50.00 | 0.06 | 0.07 | 0.11 | 0.12 | 0.08 | 0.12 | 0.18 | 0.11 |
| 52.00 | 0.05 | 0.06 | 0.11 | 0.11 | 0.08 | 0.12 | 0.16 | 0.10 |
| 57.00 | 0.05 | 0.07 | 0.11 | 0.10 | 0.07 | 0.10 | 0.17 | 0.19 |
| 59.00 | 0.05 | 0.07 | 0.10 | 0.10 | 0.07 | 0.10 | 0.15 | 0.09 |

DL - Drug Load

TABLE 6

In-vivo release of Etonogestrel from implants in humans (model) - deconvoluted from rat pharmacokinetic study

| | 15%DL Islatravir 35%DL Etonogestrel | 20%DL Islatravir 40%DL Etonogestrel | 30%DL Islatravir 30%DL Etonogestrel | 35%DL Islatravir 35%DL Etonogestrel | 15%DL Islatravir 35%DL Etonogestrel | 20%DL Islatravir 40%DL Etonogestrel | 30%DL Islatravir 30%DL Etonogestrel | 35%DL Islatravir 35%DL Etonogestrel |
|---|---|---|---|---|---|---|---|---|
| Days | Core-Diffusional Barrier | | | | Matrix | | | |
| | Daily Release, mg/day | Daily Release, mg/day | Daily Release, mg/day | Daily Release, mg/day | Daily Release, mg/day | Daily Release, mg/day | Daily Release, mg/day | Daily Release, mg/day |
| 0.041667 | 0.80 | 0.57 | 1.28 | 1.21 | 1.52 | 1.73 | 1.55 | 1.28 |
| 0.083333 | 0.87 | 0.88 | 1.46 | 1.47 | 1.75 | 1.73 | 1.75 | 1.46 |
| 0.166667 | 0.74 | 0.78 | 1.04 | 0.87 | 1.69 | 1.64 | 1.60 | 1.50 |
| 1 | 0.35 | 0.32 | 0.55 | 0.53 | 1.04 | 0.96 | 0.82 | 0.77 |
| 2 | 0.29 | 0.33 | 0.36 | 0.41 | 0.92 | 0.90 | 0.95 | 0.71 |
| 3 | 0.30 | 0.29 | 0.33 | 0.34 | 0.95 | 0.91 | 0.94 | 0.87 |
| 4 | 0.29 | 0.26 | 0.33 | 0.31 | 0.88 | 0.69 | 0.69 | 0.70 |
| 7 | 0.24 | 0.24 | 0.24 | 0.26 | 0.65 | 0.58 | 0.55 | 0.55 |
| 10 | 0.20 | 0.25 | 0.24 | 0.30 | 0.67 | 0.53 | 0.39 | 0.48 |
| 15 | 0.29 | 0.24 | 0.28 | 0.25 | 0.46 | 0.46 | 0.34 | 0.40 |
| 17 | 0.26 | 0.30 | 0.24 | 0.27 | 0.45 | 0.38 | 0.29 | 0.34 |
| 22 | 0.23 | 0.28 | 0.25 | 0.26 | 0.51 | 0.41 | 0.31 | 0.39 |
| 24 | 0.25 | 0.26 | 0.24 | 0.26 | 0.41 | 0.38 | 0.27 | 0.24 |
| 29 | 0.23 | 0.29 | 0.24 | 0.27 | 0.43 | 0.32 | 0.26 | 0.22 |
| 31 | 0.25 | 0.23 | 0.27 | 0.24 | 0.36 | 0.29 | 0.24 | 0.18 |
| 36 | 0.25 | 0.28 | 0.27 | 0.27 | 0.28 | 0.27 | 0.20 | 0.16 |
| 38 | 0.25 | 0.25 | 0.24 | 0.26 | 0.32 | 0.31 | 0.18 | 0.12 |
| 43 | 0.21 | 0.26 | 0.24 | 0.25 | 0.33 | 0.30 | 0.18 | 0.15 |
| 45 | 0.18 | 0.26 | 0.21 | 0.25 | 0.28 | 0.28 | 0.19 | 0.13 |
| 50 | 0.18 | 0.24 | 0.20 | 0.25 | 0.28 | 0.27 | 0.18 | 0.12 |
| 52 | 0.18 | 0.25 | 0.21 | 0.26 | 0.26 | 0.29 | 0.18 | 0.13 |
| 57 | 0.20 | 0.25 | 0.22 | 0.26 | 0.23 | 0.24 | 0.20 | 0.12 |
| 59 | 0.17 | 0.21 | 0.23 | 0.25 | 0.23 | 0.22 | 0.15 | 0.14 |

### Example 5

### Preparation of Implant Drug Delivery Systems Containing 20 wt% Islatravir, 40% Etonogestrel, and a Radiopaque Agent

Matrix implants were prepared using an extrusion injection molded process. Hydrophobic, aliphatic thermoplastic polyurethane, islatravir, anhydrate form and etonogestrel, were blended with 20 wt% ISL, 40 wt% ETO and 10 wt% Barium Sulfate as a radiopaque agent. The preblend was compounded with a twin screw extruder at temperatures ranging from 150-160° C., screw speed at 50-60 rpm, torque at 20-40 Ncm, and then re-granulated and mixed. The mix was melt extruded with a twin screw extruder at temperatures ranging from 150-160° C., screw speed at 50-60 rpm and torque at 20-90 Ncm to form a 1.8±0.1 mm diameter filament. Extruded filaments were cut to length 50±2 mm and compression molded at 150-160° C. and 10-20 tons. Preformed filaments were then trimmed of flash and cut to final length 40±2 mm to generate combination implants of islatravir+ etonogestrel in thermoplastic polyurethane.

Reservoir implants were prepared using already formed matrix filaments as described above and pre-extruded hydrophilic, swelling thermoplastic polyurethane of 5% or 10% nominal water uptake tubing for the diffusional barrier . Extruded tubing was placed over pre-formed filaments of core material then samples with diffusional barriers were placed in an over-molding machine and compression molded at 180-190° C. and 8-12 tons to bond the core and diffusional barrier, and cut to final length 40±2 mm.

### Counterexample 1

### Preparation of Implant Drug Delivery Systems Containing 70 wt% Islatravir Anhydrate and a Radiopaque Agent

The milled polymer, and islatravir, anhydrate form, were blended at 70 wt% ISL in hydrophobic, aliphatic thermoplastic polyurethane and 10 wt% Barium Sulfate as a radiopaque agent. The preblend was melt extruded with a twin screw extruder at temperatures ranging from 100-180° C., screw speed at 20-30 rpm, but could not be successfully processed due to high die pressure and screw torque.

### Counterexample 2

### Preparation of Implant Drug Delivery Systems Containing 60 wt% Islatravir Monohydrate and a Radiopaque Agent

The milled polymer, and islatravir, monohydrate form, were blended at 60 wt% ISL in hydrophobic, aliphatic thermoplastic polyurethane and 10 wt% Barium Sulfate as a radiopaque agent. The preblend was melt extruded with a twin screw extruder at temperatures ranging from 100-180° C., screw speed at 20-30 rpm, but could not be successfully processed due to high die pressure and screw torque.

### Counterexample 3

### Preparation of Implant Drug Delivery Systems Containing 60 wt%Islatravir Monohydrate and a Radiopaque Agent With EVA 28 + Hydrophilic Tpu Diffusional Barrier

Implants were prepared using an extrusion process. The milled polymer, and islatravir, monohydrate form, were blended at 60 wt% ISL in polyethylene vinyl acetate, 28% vinyl acetate (EVA 28) and 10 wt% Barium Sulfate as a radiopaque agent. The preblend was melt extruded with a twin screw extruder at temperatures ranging from 100-160° C., screw speed at 20-30 rpm, and then pelletized. The pellets were then sieved and lubricated, then formed the core inside a diffusional barrier of hydrophilic, swelling thermoplastic polyurethane of 5% nominal water uptake prepared by co-extrusion with two single-screw extruders with temperatures ranging from 130-160° C., and screw speed at 20-25 rpm to form a 2±0.05 mm diameter filament, with 0.05 - 0.25 mm diffusional barrier thickness, and then cut to a length of 40±2 mm. Upon soaking these samples in phosphate buffer saline, the diffusional barriers expanded and delaminated in some cases, perhaps due to insufficient adhesion between the core and diffusional barrier.

What is claimed is:

1. An implant drug delivery system comprising:
(a) a core comprising a biocompatible nonerodible polymer and (i) islatravir anhydrate, which is present in the core between 10% to 50% by weight, and (ii) etonogestrel, which is present in the core between 25% to 50% by weight, and
(b) a biocompatible nonerodible diffusional barrier comprising a polymer,
wherein said diffusional barrier has a thickness between 50 µm and 300 µm, wherein said implant drug delivery system is implanted subdermally and (i) islatravir anhydrate is continually released in vivo at a rate resulting in a plasma concentration of islatravir between 0.02 ng/mL and 300.0 ng/mL for a period of six months to thirty-six months, and (ii) etonogestrel is continually released in vivo at a rate resulting in a plasma concentration of etonogestrel between 0.15 ng/mL and 1.2 ng/mL a period of six months to thirty-six months.

2. The implant drug delivery system of claim 1 wherein the islatravir plasma concentration is between 0.02 ng/mL and 30.0 ng/mL.

3. The implant drug delivery system of claim 2 wherein the islatravir plasma concentration is between 0.02 ng/mL and 8.0 ng/mL.

4. The implant drug delivery system of claim 1 wherein the islatravir anhydrate is present in the core at 15% by weight.

5. The implant drug delivery system of claim 1 wherein the islatravir anhydrate is present in the core at 20% by weight.

6. The implant drug delivery system of claim 1 wherein the islatravir anhydrate is present in the core at 30% by weight.

7. The implant drug delivery system of claim 1 wherein the islatravir anhydrate is present in the core at 35% by weight.

8. The implant drug delivery system of claim 1 wherein the etonogestrel is present in the core at 30% by weight.

9. The implant drug delivery system of claim 1 wherein the etonogestrel is present in the core at 35% by weight.

10. The implant drug delivery system of claim 1 wherein the etonogestrel is present in the core at 40% by weight.

11. The implant drug delivery system of claim 1 wherein the biocompatible nonerodible polymer in the core is selected from the group consisting of ethylene vinyl acetate copolymer (EVA), poly(urethane), silicone, crosslinked poly(vinyl alcohol), poly(hydroxy ethylmethacrylate), acyl substituted cellulose acetates, partially hydrolyzed alkylene-vinyl acetate copolymers, completely hydrolyzed alkylene-vinyl acetate copolymers, unplasticized polyvinyl chloride, crosslinked homopolymers of polyvinyl acetate, crosslinked copolymers of polyvinyl acetate, crosslinked polyesters of acrylic acid, crosslinked polyesters of methacrylic acid, polyvinyl alkyl ethers, polyvinyl fluoride, polycarbonate, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, chlorosulphonated polylefins, and combinations thereof.

12. The implant drug delivery system of claim 11 wherein the biocompatible nonerodible polymer in the core comprises poly(urethane).

13. The implant drug delivery system of claim 1, wherein the diffusional barrier comprises a hydrophilic polymer or a hydrophobic polymer with a soluble filler.

14. The implant drug delivery system of claim 13, wherein the diffusional barrier comprises a polymer selected from the group consisting of ethylene vinyl acetate copolymer (EVA), silicone, crosslinked poly(vinyl alcohol), unplasticized polyvinyl chloride, crosslinked homopolymers of polyvinyl acetate, crosslinked copolymers of polyvinyl acetate, crosslinked polyesters of acrylic acid, crosslinked polyesters of methacrylic acid, polyvinyl alkyl ethers, polyvinyl fluoride, polycarbonate, polyamide, polysulphones, styrene acrylonitrile copolymers, crosslinked poly(ethylene oxide), poly(alkylenes), poly(vinyl imidazole), poly(ethylene terephthalate), poly(urethane), poly(hydroxy ethylmethacrylate), acyl substituted cellulose acetates, partially hydrolyzed alkylene-vinyl acetate copolymers, completely hydrolyzed alkylene-vinyl acetate copolymers, poly(esters), polyphosphazenes, chlorosulphonated polylefins, combinations thereof,.

15. The implant drug delivery system of claim 14, wherein the diffusional barrier comprises a polymer selected from the group consisting of poly(urethane), poly(hydroxy ethylmethacrylate), acyl substituted cellulose acetates, partially hydrolyzed alkylene-vinyl acetate copolymers, completely hydrolyzed alkylene-vinyl acetate copolymers, poly(esters), polyphosphazenes, chlorosulphonated polylefins, and combinations thereof.

16. The implant drug delivery system of claim 15, wherein the diffusional barrier comprises poly(urethane).

17. The implant drug delivery system of claim 16, wherein the poly(urethane) has a water uptake of between 1% and 20% by weight.

18. The implant drug delivery system of claim 13 wherein the diffusional barrier further comprises an additive selected from the group consisting of polyethylene glycol, citric acid, and poloxamer.

19. The implant drug delivery system of claim 1 wherein the diffusional barrier has a thickness between 100 µm and 200 µm.

20. The implant drug delivery system of claim 1 wherein the core and the biocompatible nonerodible diffusional barrier both comprise poly(urethane).

21. The implant drug delivery system of claim 1, wherein the core and the biocompatible nonerodible diffusional barrier are prepared by co-extrusion, and the co-extrusion is carried out at a temperature between 130° C. and 190° C.

22. The implant drug delivery system of claim 21 wherein the co-extrusion is carried out at a temperature between 130° C. and 160° C.

23. The implant drug delivery system of claim 1, wherein the core and the biocompatible nonerodible diffusional barrier are prepared by injection molding, and the injection molding is carried out at a temperature between 130° C. and 190° C.

24. The implant drug delivery system of claim 23 wherein the injection molding is carried out at a temperature between 150° C. and 160° C.

25. The implant drug delivery system of claim 1 wherein the islatravir anhydrate is characterized by a powder x-ray diffraction pattern with at least peaks at diffraction angles degrees 2 theta (+/- 0.2°) 11.79, 12.39, 14.70 and 15.51 in a powder x-ray diffraction obtained using Cu K alpha radiation.

26. The implant drug delivery system of claim 1 comprising between 1% and 20% by weight of a radiopaque material.

27. The implant drug delivery system of claim 1 wherein the islatravir is released at therapeutic concentrations for a duration from between twenty-four months and thirty-six months.

28. The implant drug delivery system of claim 1 wherein the etonogestrel is released at contraceptive concentrations for a duration from between twenty-four months and thirty-six months.

29. A method of treating or preventing HIV infection and preventing pregnancy with an implant drug delivery system according to claim 1.

30. An implant drug delivery system comprising:
(a) a core comprising a biocompatible nonerodible polymer and (i) islatravir, which is present in the core between 15% to 35% by weight, and (ii) etonogestrel, which is present in the core between 30% to 40% by weight, and optionally comprises
(b) a biocompatible nonerodible diffusional barrier comprising a polymer,
wherein said diffusional barrier has a thickness between 50 µm and 300 µm, and wherein at day 30, (i) islatravir is released in vitro at a rate resulting in a concentration at of islatravir between 0.020 mg and 0.050 mg, and (ii) etonogestrel is released in vitro at a rate resulting in a concentration of etonogestrel between 0.025 mg and 0.050 mg.
